(19) United States
(12) Patent Application Publication
(10) Pub. No.: US 2011/0212980 A1
(43) Pub. Date: Sep. 1, 2011

(54) COMBINATIONS FOR THE TREATMENT OF MIGRAINE

(75) Inventors: Graeme Bilbe, Neuchatel (CH); Baltazar Gomez-Mancilla, Basel (CH); Daniel Hoyer, Huningue (FR); Donald Johns, Woburn, MA (US); Hans O. Kalkman, Basel (CH); Kevin Hall Mcallister, Buschwiller (FR)

(73) Assignee: NOVARTIS AG

(21) Appl. No.: 13/125,331

(22) PCT Filed: Oct. 21, 2009
(86) PCT No.: PCT/EP2009/063812

§ 371 Date: May 3, 2011

Related U.S. Application Data
(60) Provisional application No. 61/107,547, filed on Oct. 22, 2008.

(30) Foreign Application Priority Data
Oct. 22, 2008 (EP) 08167250.3

(51) Int. Cl. A61K 31/517; A61P 25/06

(52) U.S. Cl. 514/266.23; 514/266.3; 514/266.24

(57) ABSTRACT

The present invention relates to novel combinations suitable for the treatment of migraine of various genesis or aetiology, which comprise, as active ingredients, at least one 5-HT_{1 B/1 D} receptor agonist and at least one selective, competitive AMPA receptor antagonist being a 1 H-quinazoline-2,4-dione of formula (I) as defined in the claims; to their preparation; to their use as medicaments and to medicaments comprising them.

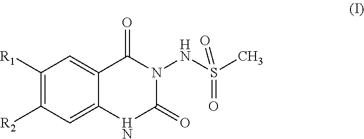

The present invention relates to combinations, which comprise at least one 5-HT_{1B/1D} receptor agonist and at least one competitive AMPA receptor antagonist, being a 1H-quinazoline-2,4-dione, to pharmaceutical compositions comprising them, and to their use as medicaments for migraine therapy.

Migraine is a chronic, episodic and debilitating clinical condition having a high prevalence (5-15%) and socioeconomic impact. For many patients, therapy is still unsatisfactory.

Migraine can be divided into two major subtypes: migraine without aura (MO) and migraine with aura (MA). MO is a clinical syndrome characterized by headache attacks lasting 4-72 hours. Typical characteristics of the headache are unilateral location, pulsating quality (throbbing), moderate or severe intensity, aggravation by physical activity (which causes a mechanical strain on meningeal blood vessels) and association with nausea, vomiting, photophobia and/or phonophobia. About 70% of migraineurs have MO. In MA, attacks are accompanied by reversible focal neurological symptoms (mostly visual, but also sensory or motor symptoms). Aura develops over 5-20 minutes and lasts for less than 60 minutes. Headache with the features of MO usually follows the aura. About 30% of migraineurs have MA.

Other, less common, types of migraine exist and include migraine with prolonged aura (aura symptoms last longer than 60 minutes); migraine aura without headache; migraine with acute onset aura; basilar migraine which can be associated with vertigo, gait perturbances and/or loss of consciousness; ophthalmoplegic migraine associated with ocular paralysis, diplopia and/or ptosis; retinal migraine; end familial hemiplegic migraine associated with hemiparesis or hemiplegia.

Pharmacological interventions for the management of migraine can be categorized into two general strategies: prevention of pain and/or associated symptomology and treatment to relieve/stop the pain and associated symptomology.

The objective of the preventive (prophylactic) therapy is to reduce the frequency of the migraine attacks, reduce the severity and/or shorten the duration of the attacks. Agents useful for migraine preventive therapy include anticonvulsants, antidepressants, beta blockers, calcium channel blockers nonsteroidal anti-inflammatory drugs (NSAIDs), and serotonin receptor antagonists. Many of these agents are used off-label in migraine prophylaxis.

Agents useful for migraine treatment include 5-HT_{1B/1D} receptor agonists (e.g. “triptans”), antiemetics, ergot derivatives and analgesics, e.g. NSAIDs.

5-HT_{1B/1D} receptor agonists (e.g. triptans, such as sumatriptan) are very common in migraine treatment as they are suitable for many, but not all, migraineurs. Usually, triptans can reduce the symptoms/abort the attack within 30-90 minutes in 70-80% of patients. Triptans also have drawbacks, as they have potential for unwanted side-effects at therapeutic doses. The most common adverse effects are the recurrence of migraine and cardiovascular side effects, the later could be so debilitating to stop migraine treatment or even life threatening (e.g. excessive coronary constriction). Furthermore, the 5-HT_{1B/1D} receptor agonists are not active in migraine prevention.

Combinations of 5-HT_{1B/1D} receptor agonists and co-drugs for the treatment of migraine are known and described, for example, in WO 03/015787 and WO 06/072413 (co-drugs: calcitonin gene-related peptide antagonists); WO 03/072138 (co-drugs: anticonvulsants); WO 06/027681 (co-drugs: GABA analogues); and WO 07/127207 (co-drug: diclofenac).

Certain glutamate receptor antagonists, which show a variable degree of selectivity towards the AMPA-receptor and act on different binding sites at the receptor, are under clinical evaluation as migraine treatments.

The non-selective, competitive AMPA/GluR5 receptor antagonist LY293558 (tezampanel) is currently evaluated (J Striessnig, Durg Discovery Today: Disease Mechanisms, 2005, 2(4), 453-462; CN Sang et al, Cephalalgia, 2004, 24, 596-602); the relevance of the AMPA-versus GluR5-antagonism of this compound is still under discussion, evidence exists favoring a GluR5-mediated mechanism of action (B. Weiss et al, Journal of Pharmacology and Experimental Therapeutics, 2006, 318, 772-781).

Furthermore, the selective, non-competitive AMPA receptor antagonist E2007 (perampanel) is evaluated (J Striessnig, Durg Discovery Today: Disease Mechanisms, 2005, 2(4)), being a non-competitive antagonist, E2007 acts at an allosteric modulatory site of the receptor.

Although said anti-migraine agents are known, there is an ongoing need to provide better therapies for migraine that are, for example, more effective at lower doses, effective against a wider spectrum of migraine conditions, less prone to produce side effects (such as cardiovascular side effects), faster acting and/or longer acting. A lower rate of recurrence is also desirable.

Surprisingly, a combination therapy of a 5-HT_{1B/1D} receptor agonist and a 1H-quinazoline-2,4-dione of formula (I), being a selective, competitive AMPA receptor antagonist, offers significant benefits in migraine therapy.

The invention therefore provides a combination suitable for migraine therapy, which comprises

(A) at least one 5-HT_{1B/1D} receptor agonist as the first active ingredient; and
(B) at least one 1H-quinazoline-2,4-dione of formula (I)

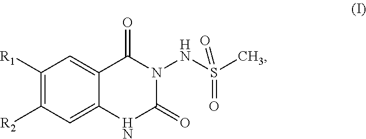

wherein
R₁ is C₁-C₆alkyl substituted by one, two or three substituents selected from hydroxy, C₁-C₆alkoxy or C₅-C₆cycloalkoxy; or

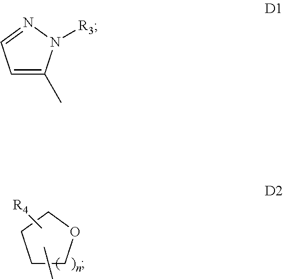

R₃ is C₁-C₆alkyl, hydroxy or C₁-C₆alkoxy-C₁-C₆alkyl;
R₄ is hydrogen or C₁-C₆alkyl;
n is 1 or 2; and
R₂ is C₁-C₃alkyl or C₁-C₃fluoroalkyl; as the second active ingredient; and in which the active ingredients are present in each case in free form or in the form of a pharmaceutically acceptable salt.

The term “5-HT_{1B/1D} receptor agonist” as used herein preferably refers to the well-known family of tryptamine-based drugs used in the treatment of migraine, commonly also known as “triptans”. 5-HT_{1B/1D} receptor agonists include almotriptan (“Axert”™, Almogran”™), eletriptan (“Relpax”™), frovatriptan (“Frova”™, “Migard”™), naratriptan (“Amerge”™, “Naramig”™) rizatriptan (“Maxalt”™), sumatriptan (“Imitrex”™, “Imigran”™) and zolmitriptan (“Zomig”™)

Further “5-HT_{1B/1D} receptor agonists” are, for example, ergotamine and dihydroergotamine.

The structure/chemical nature of an active ingredient identified by a generic or trade name may be taken from the current edition of a standard compendium, e.g. “The Merck Index”, or from a database, e.g. Patents International (e.g. IMS World Publications). The corresponding contents thereof are herewith incorporated hereinto by reference. Any person skilled in the art is fully enabled to identify the active ingredients based on these references.

Preferably the 5-HT_{1B/1D} receptor agonist is selected from the group consisting of almotriptan, eletriptan, frovatriptan, naratriptan, rizatriptan, sumatriptan and zolmitriptan.

In one embodiment, the 5-HT_{1B/1D} receptor agonist is almotriptan.

In one embodiment, the 5-HT_{1B/1D} receptor agonist is eletriptan.

In one embodiment, the 5-HT_{1B/1D} receptor agonist is frovatriptan.

In one embodiment, the 5-HT_{1B/1D} receptor agonist is naratriptan.

In one embodiment, the 5-HT_{1B/1D} receptor agonist is rizatriptan.

In one embodiment, the 5-HT_{1B/1D} receptor agonist is sumatriptan.

In one embodiment, the 5-HT_{1B/1D} receptor agonist is zolmitriptan.

The 1H-quinazoline-2,4-diones of formula (I) are selective, competitive AMPA receptor antagonists. Their manufacture and use is known from WO 06/108591, which is incorporated herein by reference. The term “selective AMPA receptor antagonists” in connection with the 1H-quinazoline-2,4-diones of formula (I) means that the compounds typically display a higher affinity towards AMPA-receptors compared with NMDA- or KA-receptors.

In the present specification, the following definitions shall apply if no specific other definition is given:

“C₁-C₆alkyl ” represents a straight-chain or branched-chain alkyl group; for example methyl, ethyl, n- or iso-propyl, n-, iso-, sec- or tert-butyl, n-pentyl, n-hexyl, with particular preference given to methyl, ethyl, n-propyl and iso-propyl.
“C₅-C₆cycloalkyl” represents cyclopentyl or cyclohexyl; preferably cyclopentyl.

Each alkyl/cycloalkyl-part of “alkoxy”, “cycloalkoxy”, “alkoxyalkyl” and “fluoroalkyl” shall have the same meaning as described in the above-mentioned definitions of “alkyl”/“cycloalkyl”. “C₁-C₃fluoroalkyl” preferably represents trifluoromethyl, difluoromethyl or fluoromethyl.

On account of asymmetrical carbon atom(s) that may be present in the 1H-quinazoline-2,4-diones of formula (I) and their salts, the compounds may exist in optically active form or in form of mixtures of optical isomers, e.g. in form of racemic mixtures or diastereomeric mixtures. All optical isomers and their mixtures, including racemic mixtures, are part of the present invention.

In one embodiment of the invention, the 1H-quinazoline-2,4-dione of formula (I) is a compound, wherein R₁ is C₁-C₆alkyl substituted by one, two or three substituents selected from hydroxy, C₁-C₆alkoxy or C₅-C₆cycloalkoxy; and R₂ is C₁-C₃alkyl or C₁-C₃fluoroalkyl.

In one embodiment of the invention, the 1H-quinazoline-2,4-dione of formula (I) is a compound, wherein R₁ is

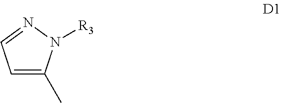

R₃ is C₁-C₆alkyl, hydroxy or C₁-C₆alkoxy-C₁-C₆alkyl; and R₂ is C₁-C₃alkyl or C₁-C₃fluoroalkyl.

In one embodiment of the invention, the 1H-quinazoline-2,4-dione of formula (I) is a compound, wherein R₁ is

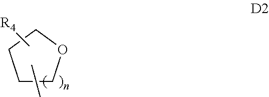

R₄ is hydrogen or C₁-C₆alkyl; n is 1 or 2; and R₂ is C₁-C₃alkyl or C₁-C₃fluoroalkyl.

In one embodiment of the invention, the 1H-quinazoline-2,4-dione of formula (I) is a compound selected from the group consisting of

A-1: N-[6-(1-Hydroxy-ethyl)-2,4-dioxo-7-trifluoromethyl-1,4-dihydro-2H-quinazolin-3-yl]-methanesulfonamide;
A-2: N-[6-(1-Methoxy-ethyl)-2,4-dioxo-7-trifluoromethyl-1,4-dihydro-2H-quinazolin-3-yl]-methanesulfonamide;
A-3: N-[6-(1-Hydroxy-propyl)-2,4-dioxo-7-trifluoromethyl-1,4-dihydro-2H-quinazolin-3-yl]-methanesulfonamide;
A-4: N-[6-(1-Isopropoxy-ethyl)-2,4-dioxo-7-trifluoromethyl-1,4-dihydro-2H-quinazolin-3-yl]-methanesulfonamide;
A-5: N-[6-(1-Ethoxy-ethyl)-2,4-dioxo-7-trifluoromethyl-1,4-dihydro-2H-quinazolin-3-yl]-methanesulfonamide;
A-6: N-[2,4-Dioxo-6-(1-propoxy-propyl)-7-trifluoromethyl-1,4-dihydro-2H-quinazolin-3-yl]-methanesulfonamide;
A-7: N-[6-(1-isopropoxy-propyl)-2,4-dioxo-7-trifluoromethyl-1,4-dihydro-2H-quinazolin-3-yl]-methanesulfonamide;
A-8: N-[7-Difluoromethyl-6-(1-ethoxy-ethyl)-2,4-dioxo-1,4-dihydro-2H-quinazolin-3-yl]-methanesulfonamide;
A-9: N-[2,4-Dioxo-6-(1-propoxy-ethyl)-7-trifluoromethyl-1,4-dihydro-2H-quinazolin-3-yl]-methanesulfonamide;
A-10: N-[6-(1-Butoxy-ethyl)-2,4-dioxo-7-trifluoromethyl-1,4-dihydro-2H-quinazolin-3-yl]-methanesulfonamide;
A-11: N-[6-(1-Isobutoxy-ethyl)-2,4-dioxo-7-trifluoromethyl-1,4-dihydro-2H-quinazolin-3-yl]-methanesulfonamide;
A-12: N-[6-(1-methoxy-butyl)-2,4-dioxo-7-trifluoromethyl-1,4-dihydro-2H-quinazolin-3-yl]-methanesulfonamide;
A-13: N-[6-(1-Ethoxy-propyl)-2,4-dioxo-7-trifluoromethyl-1,4-dihydro-2H-quinazolin-3-yl]-methanesulfonamide;
A-14: N-[6-(1-Cyclopentyloxy-ethyl)-2,4-dioxo-7-trifluoromethyl-1,4-dihydro-2H-quinazolin-3-yl]-methanesulfonamide;
A-15: N-[6-(1-Hydroxy-butyl)-2,4-dioxo-7-trifluoromethyl-1,4-dihydro-2H-quinazolin-3-yl]-methanesulfonamide;
A-16: N-[6-(1-Methoxy-2-methyl-propyl)-2,4-dioxo-7-trifluoromethyl-1,4-dihydro-2H-quinazolin-3-yl]-methanesulfonamide;
A-17: N-[6-(3-Hydroxy-propyl)-2,4-dioxo-7-trifluoromethyl-1,4-dihydro-2H-quinazolin-3-yl]-methanesulfonamide;
A-18: N-[6-(1-Hydroxy-3-methoxy-propyl)-2,4-dioxo-7-trifluoromethyl-1,4-dihydro-2H-quinazolin-3-yl]-methanesulfonamide;
A-19: N-[6-(1-Hydroxy-2-methyl-propyl)-2,4-dioxo-7-trifluoromethyl-1,4-dihydro-2H-quinazolin-3-yl]-methanesulfonamide;
B-1: N-[2,4-Dioxo-6-(tetrahydro-pyran-2-yl)-7-trifluoromethyl-1,4-dihydro-2H-quinazolin-3-yl]-methanesulfonamide;
B-2: N-[2,4-Dioxo-6-(tetrahydro-furan-2-yl)-7-trifluoromethyl-1,4-dihydro-2H-quinazolin-3-yl]-methanesulfonamide;
B-3: N-[2,4-Dioxo-6-(tetrahydro-furan-3-yl)-7-trifluoromethyl-1,4-dihydro-2H-quinazolin-3-yl]-methanesulfonamide;
C-1: N-[7-Isopropyl-6-[2-(2-methoxy-ethyl)-2H-pyrazol-3-yl]-2,4-dioxo-1,4-dihydro-2H-quinazolin-3-yl]-methanesulfonamide;
C-2: N-[6-(2-Isopropyl-2H-pyrazol-3-yl)-2,4-dioxo-7-trifluoromethyl-1,4-dihydro-2H-quinazolin-3-yl]-methanesulfonamide;
C-3: N-[7-Fluoromethyl-6-(2-isopropyl-2H-pyrazol-3-yl)-2,4-dioxo-1,4-dihydro-2H-quinazolin-3-yl]-methanesulfonamide;
C-4: N-[6-[2-(2-Methoxy-ethyl)-2H-pyrazol-3-yl]-2,4-dioxo-7-trifluoromethyl-1,4-dihydro-2H-quinazolin-3-yl]-methanesulfonamide;
C-5: N-[6-(2-Hydroxy-2H-pyrazol-3-yl)-2,4-dioxo-7-trifluoromethyl-1,4-dihydro-2H-quinazolin-3-yl]-methanesulfonamide;
C-6: N-[7-Ethyl-6-(2-isopropyl-2H-pyrazol-3-yl)-2,4-dioxo-1,4-dihydro-2H-quinazolin-3-yl]-methanesulfonamide;
C-7: N-[7-Isopropyl-6-(2-methyl-2H-pyrazol-3-yl)-2,4-dioxo-1,4-dihydro-2H-quinazolin-3-yl]-methanesulfonamide;
C-8: N-[7-Isopropyl-6-(2-isopropyl-2H-pyrazol-3-yl)-2,4-dioxo-1,4-dihydro-2H-quinazolin-3-yl]-methanesulfonamide;
C-9: N-[7-Difluoromethyl-6-(2-methyl-2H-pyrazol-3-yl)-2,4-dioxo-1,4-dihydro-2H-quinazolin-3-yl]-methanesulfonamide;
C-10: N-[7-Difluoromethyl-6-(2-isopropyl-2H-pyrazol-3-yl)-2,4-dioxo-1,4-dihydro-2H-quinazolin-3-yl]-methanesulfonamide;
C-11: N-[7-Ethyl-6-(2-methyl-2H-pyrazol-3-yl)-2,4-dioxo-1,4-dihydro-2H-quinazolin-3-yl]-methanesulfonamide;
C-12: N-[7-Ethyl-6-(2-ethyl-2H-pyrazol-3-yl)-2,4-dioxo-1,4-dihydro-2H-quinazolin-3-yl]-methanesulfonamide;
C-13: N-[7-Fluoromethyl-6-(2-methyl-2H-pyrazol-3-yl)-2,4-dioxo-1,4-dihydro-2H-quinazolin-3-yl]-methanesulfonamide;
C-14: N-[7-(1-fluoro-ethyl)-6-(2-methyl-2H-pyrazol-3-yl)-2,4-dioxo-1,4-dihydro-2H-quinazolin-3-yl]-methanesulfonamide;
C-15: N-[7-(1,1-difluoro-ethyl)-6-(2-methyl-2H-pyrazol-3-yl)-2,4-dioxo-1,4-dihydro-2H-quinazolin-3-yl]-methanesulfonamide;
C-16: N-[7-(1,1-difluoro-ethyl)-6-(2-isopropyl-2H-pyrazol-3-yl)-2,4-dioxo-1,4-dihydro-2H-quinazolin-3-yl]-methanesulfonamide;
C-17: N-[7-(1-fluoro-ethyl)-6-(2-isopropyl-2H-pyrazol-3-yl)-2,4-dioxo-1,4-dihydro-2H-quinazolin-3-yl]-methanesulfonamide; and
C-18: N-[6-(2-Methyl-2H-pyrazol-3-yl)-2,4-dioxo-7-trifluoromethyl-1,4-dihydro-2H-quinazolin-3-yl]-methanesulfonamide.

In one embodiment of the invention, the 1H-quinazoline-2,4-dione of formula (I) is a compound selected from the group consisting of compound A-1, A-2, A-3, A-4, A-5, A-6, A-7, A-8, A-9, A-10, A-11, A-12, A-13, A-14, A-15, A16, A17, A-18 and A-19.

In one embodiment of the invention, the 1H-quinazoline-2,4-dione of formula (I) is a compound selected from the group consisting of compound B-1, B-2 and B-3.

In one embodiment of the invention, the 1H-quinazoline-2,4-dione of formula (I) is a compound selected from the group consisting of compound C-1, C-2, C-3, C-4, C-5, C-6, C-7, C-8, C-9, C-10, C-11, C-12, C-13, C-14, C-15, C-16, C-17 and C-18.

It will be understood, that a reference to an active ingredient includes said active ingredient in free form and in form of a pharmaceutically acceptable salt. If an active ingredient has, e.g., at least one basic center, it can form an acid addition salt. An active ingredient having at least one acidic group can form a salt with a base. An active ingredient, in free form or in pharmaceutically acceptable salt form, may be in the form of a hydrate and/or may include other solvents, for example solvents used for the crystallization of a compound in solid form.

A “pharmaceutically acceptable salt” is intended to mean a salt of a free base/free acid of an active ingredient that is not toxic, biologically intolerable, or otherwise biologically undesirable. Preferred pharmaceutically acceptable salts are those that are pharmacologically effective and suitable for contact with the tissues of patients without undue toxicity, irritation, or allergic response. Such salts are known in the field (e.g. S M Berge et al, “Pharmaceutical Salts”, J Pharm Sd, 1977, 66:1-19; and “Handbook of Pharmaceutical Salts, Properties, Selection, and Use”, R H Stahl, C G Wermuth, Eds, Wiley-VCH and VHCA: Zurich, 2002).

In one embodiment of the invention, the 1H-quinazoline-2,4-diones of formula (I) are used in free form.

The term “migraine” as used herein includes, but is not limited to, migraine with aura and migraine without aura.

A combination, which comprises (A) at least one 5-HT_{1B/1D} receptor agonist as the first active ingredient and (B) at least one 1H-quinazoline-2,4-dione of formula (I) as the second active ingredient, and in which the active ingredients are present in each case in free form or in the form of a pharmaceutically acceptable salt, will be referred to hereinafter as a “COMBINATION OF THE INVENTION”.

Further, the invention relates to a COMBINATION OF THE INVENTION, such as a combined preparation or pharmaceutical composition, for simultaneous, separate or sequential use.

The term “combined preparation” as used herein defines especially a “kit of parts” in the sense, that the first and the second active ingredient as defined above can be dosed independently, either in separate form or by use of different fixed combinations with distinguished amounts of the active ingredients. The ratio of the amount of the active ingredient (A) to the amount of the active ingredient (B) to be administered in the combined preparation can be varied, e.g. in order to cope with the needs of a patient sub-population to be treated or the needs of a single patient, which needs can be different due to age, sex, body weight, etc. of a patient. The parts of the kit of parts can be administered simultaneously or chronologically staggered, e.g. at different time points and with equal or different time intervals for any part of the kit of parts.

Surprisingly, the administration of a COMBINATION OF THE INVENTION results in a beneficial, for example synergistic, therapeutic effect or in other surprising beneficial effects, for example fewer and/or weaker side effects, compared to a monotherapy applying only one of the active ingredients used in the COMBINATION OF THE INVENTION.

In particular, a COMBINATION OF THE INVENTION, which comprises subeffective doses of a 5-HT_{1B/1D} receptor agonist and of a 1H-quinazoline-2,4-dione of formula (I) may achieve the same effect as effective doses of either compound alone.

In particular, in patients non-responding to a 5-HT_{1B/1D} receptor agonist, a COMBINATION OF THE INVENTION, may achieve a higher therapeutic effect compared to a monotherapy with a 1H-quinazoline-2,4-dione of formula (I) alone.

In particular, in patients non-responding to a 1H-quinazoline-2,4-dione of formula (I), a COMBINATION OF THE INVENTION, may achieve a higher therapeutic effect compared to a monotherapy with a 5-HT_{1B/1D} receptor agonist alone.

A further benefit is, that lower doses of the active ingredients of the COMBINATION OF THE INVENTION can be used, compared to a monotherapy applying only one of the active ingredients used in the COMBINATION OF THE INVENTION. For example, the dosages used may not only be smaller, but may also be applied less frequently. Also, the incidence of side effects may be diminished and/or the responder rate to migraine therapies based on 5-HT_{1B/1D} receptor agonists or 1H-quinazoline-2,4-diones of formula (I) may be higher. All of this is in accordance with the desire and requirements of the patient to be treated.

Preferably, a COMBINATION OF THE INVENTION comprises at least one 5-HT_{1B/1D} receptor agonist, especially a 5-HT_{1B/1D} receptor agonist selected from the group consisting of sumatriptan, naratriptan, rizatriptan, zolmitriptan, eletriptan, frovatriptan and almotriptan, and at least one 1H-quinazoline-2,4-dione of formula (I), preferably, a compound selected from the group consisting of compound A-1, A-2, A-3, A-4, A-5, A-6, A-7, A-8, A-9, A-10, A-11, A-12, A-13, A-14, A-15, A16, A17, A-18, A-19, B-1, B-2, B-3, C-1, C-2, C-3, C-4, C-5, C-6, C-7, C-8, C-9, C-10, C-11, C-12, C-13, C-14, C-15, C16, C17 and C-18.

In one embodiment, the COMBINATION OF THE INVENTION comprises at least almotriptan, and a compound selected from the group consisting of compound A-1, A-2, A-3, A-4, A-5, A-6, A-7, A-8, A-9, A-10, A-11, A-12, A-13, A-14, A-15, A16, A17, A-18, A-19, B-1, B-2, B-3, C-1, C-2, C-3, C-4, C-5, C-6, C-7, C-8, C-9, C-10, C-11, C-12, C-13, C-14, C-15, C16, C17 and C-18.

In one embodiment, the COMBINATION OF THE INVENTION comprises at least eletriptan, and a compound selected from the group consisting of compound A-1, A-2, A-3, A-4, A-5, A-6, A-7, A-8, A-9, A-10, A-11, A-12, A-13, A-14, A-15, A16, A17, A-18, A-19, B-1, B-2, B-3, C-1, C-2, C-3, C-4, C-5, C-6, C-7, C-8, C-9, C-10, C-11, C-12, C-13, C-14, C-15, C16, C17 and C-18.

In one embodiment, the COMBINATION OF THE INVENTION comprises at least frovatriptan, and a compound selected from the group consisting of compound A-1, A-2, A-3, A-4, A-5, A-6, A-7, A-8, A-9, A-10, A-11, A-12, A-13, A-14, A-15, A16, A17, A-18, A-19, B-1, B-2, B-3, C-1, C-2, C-3, C-4, C-5, C-6, C-7, C-8, C-9, C-10, C-11, C-12, C-13, C-14, C-15, C16, C17 and C-18.

In one embodiment, the COMBINATION OF THE INVENTION comprises at least naratriptan, and a compound selected from the group consisting of compound A-1, A-2, A-3, A-4, A-5, A-6, A-7, A-8, A-9, A-10, A-11, A-12, A-13, A-14, A-15, A16, A17, A-18, A-19, B-1, B-2, B-3, C-1, C-2, C-3, C-4, C-5, C-6, C-7, C-8, C-9, C-10, C-11, C-12, C-13, C-14, C-15, C16, C17 and C-18.

In one embodiment, the COMBINATION OF THE INVENTION comprises at least rizatriptan, and a compound selected from the group consisting of compound A-1, A-2, A-3, A-4, A-5, A-6, A-7, A-8, A-9, A-10, A-11, A-12, A-13, A-14, A-15, A16, A17, A-18, A-19, B-1, B-2, B-3, C-1, C-2, C-3, C-4, C-5, C-6, C-7, C-8, C-9, C-10, C-11, C-12, C-13, C-14, C-15, C16, C17 and C-18.

In one embodiment, the COMBINATION OF THE INVENTION comprises at least sumatriptan, and a compound selected from the group consisting of compound A-1, A-2, A-3, A-4, A-5, A-6, A-7, A-8, A-9, A-10, A-11, A-12, A-13, A-14, A-15, A16, A17, A-18, A-19, B-1, B-2, B-3, C-1, C-2, C-3, C-4, C-5, C-6, C-7, C-8, C-9, C-10, C-11, C-12, C-13, C-14, C-15, C16, C17 and C-18.

In one embodiment, the COMBINATION OF THE INVENTION comprises at least zolmitriptan, and a compound selected from the group consisting of compound A-1, A-2, A-3, A-4, A-5, A-6, A-7, A-8, A-9, A-10, A-11, A-12, A-13, A-14, A-15, A16, A17, A-18, A-19, B-1, B-2, B-3, C-1, C-2, C-3, C-4, C-5, C-6, C-7, C-8, C-9, C-10, C-11, C-12, C-13, C-14, C-15, C16, C17 and C-18.

The invention also relates to a pharmaceutical composition comprising a COMBINATION OF THE INVENTION as active ingredients and at least one pharmaceutically acceptable carrier. In this composition, the first and the second active ingredients can be administered together, one after the other or separately, in one combined unit dosage form or in two separate unit dosage forms. The unit dosage form may also be a fixed combination.

A pharmaceutical composition according to the invention is, preferably, suitable for enteral administration, such as oral or rectal administration; or parenteral administration, such as intramuscular, intravenous, nasal or transdermal administration, to a warm-blooded animal (human beings and animals) that comprises a therapeutically effective amount of the active ingredients and one or more suitable pharmaceutically acceptable carriers.

Preferred are compositions for oral, sublingual, rectal, intravenous or nasal administration.

A composition for enteral or parenteral administration is, for example, a unit dosage form, such as a sugar-coated tablet, a tablet, a capsule, a suppository or an ampoule.

The unit content of active ingredients in an individual dose need not in itself constitute an effective amount, since such an amount can be reached by the administration of a plurality of dosage units. A composition according to the invention may contain, e.g., from about 10% to about 100%, preferably from about 20% to about 60%, of the active ingredients.

If not indicated otherwise, a pharmaceutical composition according to the invention is prepared in a manner known per se, e.g. by means of conventional mixing, granulating, sugar-coating, dissolving or lyophilizing processes. In preparing a composition for an oral dosage form, any of the usual pharmaceutical media may be employed, for example water, glycols, oils, alcohols, carriers, such as starches, sugars, or microcrystalline cellulose, diluents, granulating agents, lubricants, binders, disintegrating agents and the like. Because of their ease of administration, tablets and capsules represent the most advantageous oral dosage unit forms, in which case solid pharmaceutical carriers are obviously employed.

Furthermore, the invention relates to the use of a COMBINATION OF THE INVENTION for the treatment of migraine.

Furthermore, the invention relates to the use of a COMBINATION OF THE INVENTION for the preparation of a medicament for the treatment of migraine.

Furthermore, the invention relates to a method of treating migraine in a human, which comprises administering to the human a therapeutically effective amount of a COMBINATION OF THE INVENTION. In particular, a therapeutically effective amount of each of the active ingredients of the COMBINATION OF THE INVENTION may be administered simultaneously or sequentially in any order, and the components may be administered separately or as a fixed combination. For example, the method of treating or ameliorating may comprise (i) the administration of the first active ingredient and (ii) the administration of the second active ingredient, simultaneously or sequentially in any order, in jointly therapeutically effective amounts, preferably in synergistically effective amounts. The individual active ingredients of the COMBINATION OF THE INVENTION can be administered separately at different times during the course of the therapy or concurrently in divided or single combination forms. The instant invention is to be understood as embracing all such regimes of simultaneous or alternating administration. Furthermore, the term “administering” also encompasses the use of a prodrug of an active ingredient, that is converted in vivo into the active ingredient.

The effective dosage of each of the active ingredients employed in the COMBINATION OF THE INVENTION may vary, depending on the particular active ingredient or pharmaceutical composition employed, the mode of administration, the severity of the migraine to be treated, the age, sex, body weight, etc. of the patient, and the like. Thus, the dosage regimen for the COMBINATION OF THE INVENTION is selected in accordance with a variety of factors, including the renal and hepatic function of the patient. A physician or clinician of ordinary skill can readily determine and prescribe the appropriate dosage regimen.

The effective dosage for migraine therapy may be reached by administering one or more than one unit dosage of the COMBINATION OF THE INVENTION per day.

Unit dosage forms of the 5-HT_{1B/1D} receptor agonist typically contain
6.25-12.5 mg almotriptan p.o.;
20-40 mg eletriptan p.o.;
2.5-5 mg frovatriptan p.o.;
2.5-5 mg naratriptan p.o.;
5-10 mg rizatriptan p.o.;
20 mg sumatriptan s.c.;
50-100 mg sumatriptan p.o.;
2.5-5 mg zolmitriptan p.o.;
2-4 mg ergotamine p.o.;
2-4 mg dihydroergotamine p.o.;
2 mg dihydroergotamine intranasal.

Unit dosage forms of the 1H-quinazoline-2,4-dione of formula (I) typically contain 100-200 mg p.o.

When an active ingredient employed in the COMBINATION OF THE INVENTION is applied in the form as marketed as monotherapy in migraine, its dosage and mode of administration can take place in accordance with the information provided in the packet leaflet of the marketed product, if not mentioned otherwise herein.

Furthermore, the invention relates to a COMBINATION OF THE INVENTION for the use as a medicament.

Furthermore, the invention relates to a COMBINATION OF THE INVENTION for the treatment of migraine.

Furthermore, the invention relates to a kit comprising

(a) a 5-HT_{1B/1D} receptor agonist, in free form or in the form of a pharmaceutically acceptable salt,
(b) a 1H-quinazoline-2,4-dione of formula (I), in free form or in the form of a pharmaceutically acceptable salt,
(c) instructions for the simultaneous, separate or sequential use thereof in the treatment of migraine, and
(d) at least one container for containing components (a) and (b).
In one embodiment, the 5-HT_{1B/1D} receptor agonist is contained in a first container and the 1H-quinazoline-2,4-dione of formula (I) is contained in a second container.

Furthermore, the invention relates to a commercial package comprising a COMBINATION OF THE INVENTION as active ingredients and written instructions for the simultaneous, separate or sequential use thereof in the treatment of migraine.

Furthermore, the invention relates to:

(1) The use of a 1H-quinazoline-2,4-dione of formula (I) in the manufacture of a medicament for treating migraine, wherein said medicament is prepared for administration of a 5-HT_{1B/1D} receptor agonist.
(2) The use of a trip5-HT_{1B/1D} receptor agonist tan in the manufacture of a medicament for treating migraine, wherein said medicament is prepared for administration of a 1H-quinazoline-2,4-dione of formula (I).
(3) The use of a 1H-quinazoline-2,4-dione of formula (I) in the manufacture of a medicament for treating migraine in a human, wherein the human has previously been treated with a 5-HT_{1B/1D} receptor agonist.
(4) The use of a 5-HT_{1B/1D} receptor agonist in the manufacture of a medicament for treating migraine in a human, wherein the human has previously been treated with a 1H-quinazoline-2,4-dione of formula.
(5) A 1H-quinazoline-2,4-dione of formula (I) for treating migraine, wherein the 1H-quinazoline-2,4-dione is administered with a 5-HT_{1B/1D} receptor agonist.
(6) A 5-HT_{1B/1D} receptor agonist for treating migraine, wherein the 5-HT_{1B/1D} receptor agonist is administered with a 1H-quinazoline-2,4-dione of formula (I).

Some of the advantages of the COMBINATION OF THE INVENTION provided by the present invention may be appreciated in pre-clinical models (especially preclinical models of migraine pathophysiology or central sensitisation). Such models include:

the rat model for cutaneous allodynia induced by intracranial pain described by Burstein et a/ in Annals of Neurology, 2004, 55(1), 27-36;
the animal model of intracranial pain described by Ramadan in Proceedings of the National Academy of Sciences of the United States of America, 2003, 101 (12), 4274-9;
the rat model described by Burstein et al in Journal of Neurophysiology, 1999, 81 (2), 479-93; and
the rat model described by Burstein et al in Journal of Neurophysiology, 1998, 79(2), 964-82.

The advantages of the COMBINATION OF THE INVENTION will also be apparent from clinical measurements of efficacy. Such advantages can be seen as improved efficacy (e.g. the rate of migraine resolution) and as an improved safety profile (e.g. in the reduction in the adverse events). Such clinical studies are preferably randomized, double-blind, clinical studies in patients with migraine.

1-17. (canceled)

18. A combination, which comprises:
(A) at least one 5-HT_{1B/1D} receptor agonist as the first active ingredient; and
(B) at least one 1H-quinazoline-2,4-dione of formula (I) 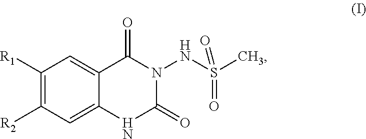
wherein
R₁ is C₁-C₆alkyl substituted by one, two or three substituents selected from hydroxy, C₁-C₆alkoxy or C₅-C₆cycloalkoxy; or 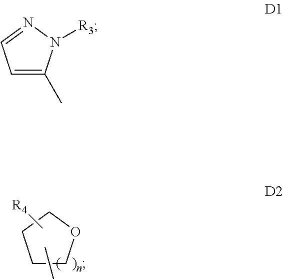
R₃ is C₁-C₆alkyl, hydroxy or C₁-C₆alkoxy-C₁-C₆alkyl;
R₄ is hydrogen or C₁-C₆alkyl;
n is 1 or 2; and
R₂ is C₁-C₃alkyl or C₁-C₃fluoroalkyl; as the second active ingredient; and
in which the active ingredients are present in each case in free form or in the form of a pharmaceutically acceptable salt.

19. A combination according to claim 18, wherein the 5-HT_{1B/1D} receptor agonist is selected from the group consisting of almotriptan, eletriptan, frovatriptan, naratriptan, rizatriptan, sumatriptan and zolmitriptan.

20. A combination according to claim 18, wherein the 5-HT_{1B/1D} receptor agonist is sumatriptan.

21. A pharmaceutical composition, which comprises a combination as defined in claim 18 as active ingredients and at least one pharmaceutically acceptable carrier.

22. A pharmaceutical composition, which comprises a combination as defined in claim 19 as active ingredients and at least one pharmaceutically acceptable carrier.

23. A pharmaceutical composition, which comprises a combination as defined in claim 20 as active ingredients and at least one pharmaceutically acceptable carrier.

24. A method of treating migraine in a human in need thereof, comprising administering to the human a therapeutically effective amount of a combination as defined in claim 18.

25. A method of treating migraine in a human in need thereof, comprising administering to the human a therapeutically effective amount of a combination as defined in claim 19.

26. A method of treating migraine in a human in need thereof, comprising administering to the human a therapeutically effective amount of a combination as defined in claim 20.

27. A method according to claim 24, wherein, before the step of administering, the human has been treated with a 5-HT_{1B/1D} receptor agonist.

28. A method according to claim 25, wherein, before the step of administering, the human has been treated with a 5-HT_{1B/1D} receptor agonist.

29. A method according to claim 26, wherein, before the step of administering, the human has been treated with a 5-HT_{1B/1D} receptor agonist.

30. A method according to claim 24, wherein, before the step of administering, the human has been treated with a 1H-quinazoline-2,4-dione of formula (I).

31. A method according to claim 25, wherein, before the step of administering, the human has been treated with a 1H-quinazoline-2,4-dione of formula (I).

32. A method according to claim 26, wherein, before the step of administering, the human has been treated with a 1H-quinazoline-2,4-dione of formula (I).

33. A method according to claim 24, wherein the step of administering comprises administering the 1H-quinazoline-2,4-dione with the 5-HT_{1B/1D} receptor agonist.

34. A method according to claim 25, wherein the step of administering comprises administering the 1H-quinazoline-2,4-dione with the 5-HT_{1B/1D} receptor agonist.

35. A method according to claim 26, wherein the step of administering comprises administering the 1H-quinazoline-2,4-dione with the 5-HT_{1B/1D} receptor agonist.
